(19) United States
(12) Patent Application Publication
(10) Pub. No.: US 2015/0224264 A1
(43) Pub. Date: Aug. 13, 2015

(54) SYRINGE CONTAINING A COMPOSITION, ESPECIALLY A PHARMACEUTICAL COMPOSITION, COMPRISING IMMUNOGLOBINS, METHOD FOR THE PRODUCTION THEREOF AND USE OF SAME

(71) Applicant: LABORATOIRE FRANCAIS DU FRACTIONNEMENT ET DES BIOTECHNOLOGIES, Les Ulis (FR)

(72) Inventor: Florence Arvis, Charbonnieres-Les-Bains (FR)

(21) Appl. No.: 14/427,088

(22) PCT Filed: Sep. 12, 2013
(86) PCT No.: PCT/FR2013/052096

§ 371 Date: Mar. 10, 2015

(30) Foreign Application Priority Data
Sep. 12, 2012 (FR) 1258580

(51) Int. Cl. A61M 5/315; A61M 5/00

(52) U.S. Cl. CPC A61M 5/31513; A61M 5/002; A61M 5/28

(57) ABSTRACT

A syringe containing a composition, in particular pharmaceutical, including immunoglobulins, its manufacturing method and its use.

A subject of the present invention is a syringe containing a composition, in particular pharmaceutical, comprising immunoglobulins, its manufacturing method and its use.

Numerous pathologies are currently treated with immunoglobulin compositions, in particular immunoglobulins G (IgG). There can for example be mentioned primary immune deficiencies with an antibody production defect, Kawasaki disease, childhood and adult immunologic thrombocytopaenic purpura, immunologic thrombocytopaenic purpura associated with HIV infection, secondary immune deficiencies with an antibody production defect, in particular chronic lymphoid leukaemia and myeloma, HIV infection of children associated with bacterial infections, Guillain-Barré syndrome, acquired or constitutional immunodeficiency, cortico-resistant dermatomyositis, chronic idiopathic polyradiculoneuritis, stiff-person syndrome, autoimmune erythroblastopaenia, autoimmune neutropaenia, severe or chronic Parvovirus B19 infections, acute myasthaenia, autoantibody-induced acquired anticoagulant syndrome, rheumatoid arthritis and uveitis.

The solutions of immunoglobulins on the market, in particular of immunoglobulins G, are presented in bottles, such as in particular Hizentra® from CSL Behring, Vivaglobin® from CSL Behring or also Gammanorm® from Octapharma. This presentation, which is easy to store, has the drawback of requiring the transfer of the product from the bottle to an injection syringe. The syringe is then placed on a pump allowing the product to be administered, in particular subcutaneously. It is the responsibility of practitioners and patients to find and acquire the devices (syringe and pump).

These commercial solutions have a low immunoglobulin concentration: in Hizentra®, the immunoglobulin solution concentration is 200 g/L or 20%; in Vivaglobin® and Gammanorm®, the immunoglobulin solution concentration is 160 g/L (16%) and 165 g/L (16.5%) respectively.

Thus, the purpose of the present invention is to provide a syringe allowing the storage of immunoglobulins under appropriate conditions.

Another purpose of the invention is to provide a syringe allowing the administration of immunoglobulins.

Another purpose of the invention is to provide a syringe that is easy for the patient or the practitioner to use, in particular allowing the injection of immunoglobulin compositions at high concentrations.

Another purpose of the invention is to provide a syringe allowing the injection of an immunoglobulin composition by sub-cutaneous route.

Another purpose of the invention is to provide a kit comprising a syringe and a pump, allowing the automated administration of an immunoglobulin composition.

Consequently, a subject of the invention is the use of a syringe allowing both the storage and administration of a composition, in particular pharmaceutical, comprising immunoglobulins.

By storage, is meant the preservation of said composition comprising immunoglobulins within said syringe, said preservation being such that the product exhibits physical and/or chemical stability.

The term “physical stability” refers to the reduction or absence of formation of insoluble or soluble aggregates of the dimeric, oligomeric or polymeric forms of the immunoglobulins, as well as to the reduction or absence of any structural denaturation of the molecule.

The term “chemical stability” refers to the reduction or absence of any chemical modification of the immunoglobulins during storage, in the solid state or in dissolved form, under accelerated conditions. For example, the phenomena of hydrolysis, deamination, and/or oxidization are avoided or delayed. The oxidization of the sulphur-containing amino acids is limited.

By immunoglobulins, is meant glycoproteins having an antibody function present in the soluble form in the plasma and in numerous secretions and in the membrane form as an element of the Ag receptor at the surface of the B cells (BCR). The immunoglobulins are molecules the base unit of which is a heterotetramer constituted by two heavy chains of approximately 50-70 kDa each (so-called H chains) and two light chains of approximately 25 kDa each (so-called L chains), linked to each other by intra- and intercaternary disulphide bridges.

Immunoglobulins for therapeutic use are devoid of infectious agents, aggregates or other substances capable of giving rise to intolerance such as IgA, IgM or contaminants capable of leading to a thrombogenic risk.

To the Inventors' knowledge, no pre-filled syringes exist that allow the storage under appropriate conditions, guaranteeing stability over time, and the injection of a composition comprising immunoglobulins, said composition being in particular highly viscous, the viscosity being in particular greater than 12 mPa·s, and/or having a particularly high immunoglobulin concentration, the immunoglobulin concentration being in particular greater than 200 g/l, said injection being in particular sub-cutaneous.

The use of the pre-filled syringes according to the invention allows practitioners and patients to avoid the stages of transferring the product from the bottle to a syringe for administration which, besides improving comfort of use, also limits the risks of needlestick injuries and guarantees better sterility of the product.

According to an advantageous embodiment, the present invention relates to a use in which said immunoglobulins are polyvalent immunoglobulins.

By human polyvalent immunoglobulins is meant purified polyclonal immunoglobulins concentrated from a pool of plasma from healthy individuals the minimum number of whom is of the order of one thousand. The polyvalent immunoglobulins are more than 95% constituted by IgG, the distribution of which into sub-classes is comparable to that of normal serum. Unlike the specific immunoglobulins directed against a particular antigen, they offer the entire spectrum of the antibody activities of the IgGs from a pool of healthy donors, in particular anti-hepatitis B activity.

The invention also relates to the use of an assembly constituted by a syringe and a composition, in particular pharmaceutical, comprising immunoglobulins, in particular polyvalent immunoglobulins, more particularly human immunoglobulins G,

said syringe being pre-filled with said composition, said syringe allowing both storage and administration of said composition,
said syringe also comprising a syringe barrel comprising an opening at each of its two ends, the first opening bearing means for sealing in a leakproof manner, in particular a nozzle cap, and/or means for administering said composition, in particular a needle, the second opening being sealed in a leakproof manner by sealing means, in particular a plunger head, capable of sliding inside the plunger barrel, in particular using a plunger rod, in particular linked to said sealing means capable of sliding,
said syringe barrel, said sealing and/or administration means, and said sealing means capable of sliding delimiting a volume in which said composition is contained,
the viscosity of said composition being, at 25° C. and at atmospheric pressure, comprised from 2 to 200 mPa·s.

According to an advantageous embodiment, the present invention relates to a use in which said syringe has a permeability such that the variation in weight of the syringe containing said composition is at most 5%, compared with the initial weight of said syringe.

In particular, said syringe has a permeability such that the variation in weight of the syringe containing said composition is at most 4, 3, 2 or 1%, compared with the initial weight of said syringe.

More particularly, said syringe has a permeability such that after at least 12 months' storage, the variation in weight of the syringe containing said composition is at most 5% compared with the initial weight of said syringe.

More particularly, said syringe has a permeability such that after at least 12 months' storage, the variation in weight of the syringe containing said composition is at most 4, 3, 2 or 1%, compared with the initial weight of said syringe.

Measurement of the variation in weight of said syringe is carried out in compliance with the provisions of the ICH guideline Q1A “Stability Testing of new Drug Substances and Products” for permeable products. A 5% variation in weight relative to the initial weight of the syringe is considered significant at low relative humidity (25° C.±2° C./40% RH±5% RH or 30° C.±2° C./35% RH±5% RH).

Measurement of the variation in weight of said syringe makes it possible to quantify the permeability of the syringe, i.e. the permeability of the assembly comprising the syringe barrel, the sealing means, in particular a nozzle cap, and the sealing means capable of sliding, in particular a plunger head.

According to an advantageous embodiment, the present invention relates to a use in which the molar percentage of immunoglobulins in monomer or dimer form is greater than 85%.

The molar percentage of immunoglobulins in monomer or dimer form can be determined by any technique known to a person skilled in the art, in particular by gel filtration chromatography (High Performance Size Exclusion Chromatography, HPSEC).

Besides monomers and dimers, said immunoglobulins can be in the form of polymers or fragments.

The aggregation of the immunoglobulins in the form of polymers is in particular induced by silicone.

According to an advantageous embodiment, the present invention relates to a use in which said syringe also comprises a material facilitating the movement of said sealing means capable of sliding, in particular of said plunger head, said material being in particular silicone, the leachables content, in particular the silicone content, of said composition being less than the quantities permissible in humans.

The permissible values are indicated in particular in the following documents: “Guideline on the specification limits for residues of metal catalysts or metal reagents” (EMEA/CHMP/SWP/4446/2000), Vigilance bulletin (AFSSAPS 2003) Am journ of clinical nutrition, Klein et al. 1991, Food Standards Agency UK Expert group on vitamins and minerals 2003, or also Guide pratique: Constantes et Repères Médicaux; 5th edition Maloine Ed.

In the case of silicone, the permissible values are, for said composition contained in said syringe, for example at most 6000 particles the size of which is greater than or equal to 10 μm, and/or at most 600 particles the size of which is greater than or equal to 25 μm.

The silicone particle-type leachables can be measured for example by means of a method for quantifying subvisible particles (particles greater than 2 μm, greater than 10 μm and greater than 25 μm) which can be counted by flow microscopy (MFI) on a total volume of 2 mL or by Light Obscuration (European Pharmacopoeia method on 25 mL).

According to an advantageous embodiment, the present invention relates to a use in which the immunoglobulin concentration of said composition, in particular pharmaceutical, is comprised from 100 to 300 g/L.

According to a particularly advantageous embodiment, the present invention relates to a use in which the immunoglobulin concentration of said composition, in particular pharmaceutical, is comprised from 200 to 300 g/L, preferably from 230 to 270 g/L.

According to another particularly advantageous embodiment, the present invention relates to a use in which the immunoglobulin concentration of said composition, in particular pharmaceutical, is comprised from 100 to 200 g/L

According to an advantageous embodiment, the present invention relates to a use in which the viscosity of said composition, in particular pharmaceutical, at 25° C. and under atmospheric pressure, is comprised from 2 to 200 mPa·s.

The viscosity is measured according to the procedure described in Burckbuchler et al. European Journal of Pharmaceutics and Biopharmaceutics 2010, 76, 351, in particular in paragraph 2.4.

Thus, the viscosity can be measured according to the following procedure:
rheometer used: Thermo Scientific HAAKE MARS III rheometer;
measurement geometry: Cone/plate geometry—C35/0.5° (i.e. a plate diameter of 35 mm and a cone angle of 0.5°);
the viscosity (η) is determined by measurement of the shear rate (γ) as a function of a stress (τ) to which the solution is subjected;
the sample must be at ambient temperature, this viscosity measurement is carried out at +25° C. (temperature controlled by Peltier effect).

The viscosity measurement (in mPa·s equivalent to centipoise cP) then takes place as follows:
placing the system at a temperature controlled at +25° C.;
after the cone-plate geometry is put into place, the point of contact is verified by the device (distance “0” between the cone and the plate). This stage allows greater accuracy for the gap distance;
250 μL of solution (carefully homogenized) is deposited using a pipette+cone. Water is added to a gutter system for saturating the air with humidity; this drop of product must be deposited centred on the plate and without spreading. It is important not to have any air bubbles;
the cone is lowered 0.027 mm from the plate (distance called the “gap”). At this point in time the drop of product spreads over the entire plate;
a plastic cover is placed on the assembly throughout the whole duration of the measurement in order to avoid evaporation;
the assembly is then placed at a temperature of +25° C. for 300 sec (geometry+product);
measurement commences and takes place in 3 phases:
   a continuous increase in stress from 0 to 40 Pa for 180 sec with a shear rate measurement point every second;
   a plateau at 40 Pa for 60 sec with a measurement point every second;
   a continuous decrease in stress from 40 to 0 Pa for 180 sec with a measurement point every second;
for the immunoglobulins tested, the fluid is Newtonian, therefore a straight line of the stress (Pa) is obtained as a function of the shear rate (in 1/s); by making a linear regression over all of the measurement points of the three phases the viscosity which corresponds to the slope is obtained (FIG. 1).

The viscosity of the composition comprising immunoglobulins is linked to the immunoglobulin concentration of said composition.

Thus, a viscosity of 2 mPa·s corresponds to an immunoglobulin concentration of approximately 100 g/L. A viscosity of 200 mPa·s corresponds to an immunoglobulin concentration of approximately 300 g/L.

According to a particularly advantageous embodiment, the present invention relates to a use in which the viscosity of said composition, in particular pharmaceutical, at 25° C. and under atmospheric pressure, is comprised from 12 and 200 mPa·s, preferably from 20 and 80 mPa·s.

A viscosity of 12 mPa·s corresponds to an immunoglobulin concentration of approximately 200 g/L. A viscosity of 200 mPa·s corresponds to an immunoglobulin concentration of approximately 300 g/L. A viscosity of 20 mPa·s corresponds to an immunoglobulin concentration of approximately 230 g/L. A viscosity of 80 mPa·s corresponds to an immunoglobulin concentration of approximately 270 g/L.

According to another particularly advantageous embodiment, the present invention relates to a use in which the viscosity of said composition, in particular pharmaceutical, at 25° C. and under atmospheric pressure, is comprised from 2 to 12 mPa·s.

A viscosity of 2 mPa·s corresponds to an immunoglobulin concentration of approximately 100 g/L. A viscosity of 12 mPa·s corresponds to an immunoglobulin concentration of approximately 200 g/L.

According to an advantageous embodiment, the present invention relates to a use in which the volume of said composition, in particular pharmaceutical, stored in said syringe is comprised from 1 and 50 ml, preferably from 2 and 35 ml, more preferentially from 4 and 20 ml.

A volume of 50 ml of said composition comprising immunoglobulins can correspond to the maximum dose necessary for a patient, under extreme treatment conditions.

Said volume of said composition is less than or equal to the maximum volume that said syringe can contain.

According to a particularly advantageous embodiment, the present invention relates to a use in which the volume of said composition, in particular pharmaceutical, stored in said syringe is comprised from 1 and 10 ml, preferably from 2 and 10 ml, more preferentially from 4 and 10 ml.

According to another particularly advantageous embodiment, the present invention relates to a use in which the volume of said composition, in particular pharmaceutical, stored in said syringe is comprised from 10 and 50 ml, preferably from 10 and 35 ml, more preferentially from 10 and 20 ml.

According to an advantageous embodiment, the present invention relates to a use comprising means, in particular a needle, for the administration of said composition, in particular pharmaceutical, by intradermal, intramuscular, intravenous and sub-cutaneous, in particular sub-cutaneous, route.

According to a particularly advantageous embodiment, the present invention relates to a use in which said needle has a diameter comprised from gauge 29 and gauge 23, in particular from gauge 29 and gauge 27.

The gauge characterizes the external diameter of the needle, expressing the number of identical needles that can fit into a pipe with an internal diameter of one inch. Table 1 below illustrates the correspondence between the diameter of a needle expressed as a gauge and the external diameter of said needle in mm for a few gauge values:

TABLE 1
correspondence between the diameter of a needle
expressed as a gauge and the external diameter
of said needle in mm for a few gauge values.

| Gauge | External diameter in mm |
|---|---|
| 29 | 0.3 |
| 27 | 0.4 |
| 25 | 0.5 |
| 23 | 0.6 |

According to an advantageous embodiment, the present invention relates to a use in which the volume of said composition, in particular pharmaceutical, stored in said syringe is comprised from 1 and 50 ml, preferably from 2 and 35 ml, more preferentially from 4 and 20 ml,

said use comprising means, in particular a needle, for the administration of said composition, in particular pharmaceutical, by intradermal, intramuscular, intravenous and sub-cutaneous, in particular sub-cutaneous, route.

The syringe thus makes it possible to inject a composition comprising immunoglobulins, in particular large volumes thereof, by sub-cutaneous route.

According to an advantageous embodiment, the present invention relates to a use in which:
the volume of said composition, in particular pharmaceutical, stored in said syringe is comprised from 1 and 50 ml, preferably from 2 and 35 ml, more preferentially from 4 and 20 ml,
the immunoglobulin concentration of said composition, in particular pharmaceutical, is comprised from 100 and 300 g/L, preferably from 200 and 300 g/L, more preferentially from 230 and 270 g/L,

said use comprising means, in particular a needle, for the administration of said composition, in particular pharmaceutical, by intradermal, intramuscular, intravenous and sub-cutaneous, in particular sub-cutaneous, route.

The syringe thus makes it possible to inject a composition comprising immunoglobulins, in particular large volumes thereof, in particular at high immunoglobulin concentrations, by sub-cutaneous route.

According to an advantageous embodiment, the present invention relates to a use in which said immunoglobulins are polyvalent, said polyvalent immunoglobulins being in particular human immunoglobulins G.

According to an advantageous embodiment, the present invention relates to a use in which said composition is in the form of an aqueous solution.

By aqueous solution, is meant a mixture comprising water as solvent, in particular as sole solvent, and immunoglobulins, said immunoglobulins being soluble in said solvent.

According to an advantageous embodiment, the present invention relates to a use in which said composition, in particular pharmaceutical, comprises:
human polyvalent immunoglobulins at a concentration from 100 and 300 g/L, preferably from 200 and 300 g/L, more preferentially from 230 and 270 g/L, even more preferentially approximately 250 g/L,
one or more hydrophobic amino acids, preferably glycine,
optionally a buffer, preferably sodium acetate buffer,
a non-ionic surfactant, preferably polysorbate, more preferentially polysorbate 80, or a poloxamer.

According to a particularly advantageous embodiment, the present invention relates to a use in which said composition, in particular pharmaceutical, comprises:
human polyvalent immunoglobulins at a concentration of from 100 to 300 g/L, preferably from 200 to 300 g/L, more preferentially from 230 to 270 g/L, even more preferentially of approximately 250 g/L,
glycine,
sodium acetate buffer,
polysorbate, preferably polysorbate 80, or a poloxamer.

According to an advantageous embodiment, the present invention relates to a use in which the administration of said composition, in particular pharmaceutical, is carried out in the absence of hyaluronidase.

The storage is such that it guarantees the stability of said composition comprising immunoglobulins. This stability can in particular be monitored by means of the following analyses:
evaluation of the appearance, colour, pH, osmolality;
evaluation of the aggregation (for example measurement of the turbidity, number of subvisible particles, dynamic light scattering);
evaluation of the evaporation (for example measurement of the concentrations, the extractible volume, the mass of the syringes);
evaluation of the functionality of the immunoglobulins (for example measurement of the concentration of antibodies directed against the hepatitis B surface antigen, the integrity of the Fc function, the binding to the RFcγ IIIa or CD16a receptor).

According to an advantageous embodiment, the present invention relates to a use in which said storage of said composition is such that, following said storage of said composition, the number of subvisible particles of a size greater than 10 μm is less than or equal to approximately 3000 per ml of composition.

By subvisible particles, is meant particles greater than 2 μm, greater than 10 μm and greater than 25 μm which can be counted by flow microscopy (MFI) on a total volume of 2 mL or by Light Obscuration (European Pharmacopoeia method on 25 mL). This analysis makes it possible to quantify the content of subvisible particles in addition to visual inspection (dedicated to the visible particles, i.e. greater than 50 μm).

The analysis can be carried out by flow microscopy under a BrightWELL/DPA4100 Flow Microscope in LowMag (LM) configuration (magnification ×5). The data collected are visualized and can be processed using MFI View software.

In pre-filled syringe type packaging, the subvisible particles can be exogenous particles originating from the packaging itself, such as droplets of silicone. The subvisible particles can also reflect the state of aggregation of the product.

According to an advantageous embodiment, the present invention relates to a use in which said storage of said composition is such that, following said storage of said composition, the number of subvisible particles of a size greater than 25 μm is less than or equal to approximately 300 per ml of composition.

According to an advantageous embodiment, the present invention relates to a use in which said storage of said composition is such that, following said storage of said composition, the dynamic light scattering measurement value of the composition administered is equal to that of the composition before storage, plus or minus 20%.

The dynamic light scattering measurement makes it possible to measure the sizes (hydrodynamic radius) of the objects in solution, between approximately 1 nm and 1 μm. This technique makes it possible to monitor the early phenomena of aggregation in solution, by determining the sizes of the objects in suspension. The ALV/CGS COMPACT GONIOMETER SYSTEM light scattering platform can be used for measuring light scattering in dynamic mode (DLS=dynamic light scattering) and makes it possible to determine the hydrodynamic radius HR of colloidal solutions, mainly of proteins, corresponding to diffusing objects less than approximately 1 μm. In order to control the light scattering platform, a computer with the ALV/CGS3 software can be connected to the ALV/LES-5004 panel. The wavelength used for the analysis is λ=632.80 nm. The analysis angle is fixed at 30°, 90° and 150°.

According to an advantageous embodiment, the present invention relates to a use in which said immunoglobulins are directed against the hepatitis B surface antigen.

According to a particularly advantageous embodiment, the present invention relates to a use in which said storage of said composition is such that, following said storage of said composition, the concentration of antibodies directed against the hepatitis B surface antigen is equal to that of the composition before storage, plus or minus 20%.

The assay of the antibodies directed against the hepatitis B surface antigen is an indicator of the integrity of the Fab function of the immunoglobulins present and makes it possible to monitor the biological activity of the active ingredient.

The quantitative determination of the anti-hepatitis B activity of the immunoglobulins can be carried out by the ELISA immunoenzymatic technique. The tests are then carried out using an ETI-AB-AUK-3 Anti-HBs EIA kit (CE marked), manufactured and marketed by DIASORIN. The titration corresponds to the parallel line assay model described in the European Pharmacopoeia. This titration is carried out on the ETI-MAX 3000 (DIASORIN) microplate manager.

According to an advantageous embodiment, the present invention relates to a use in which said storage of said composition is such that, following said storage of said composition, there is integrity of the Fc function of at least 60% of said immunoglobulins.

There is integrity of the Fc function of an immunoglobulin when said function is capable of binding the complement.

Said integrity of the Fc function can be measured as follows: the rubella antigen (Aalto) is bound to human red blood cells (originating from human blood of group O) then brought into contact with the Immunoglobulin preparations to be tested. After a time necessary for the formation of immune complexes, guinea pig complement (Tebu-Bio Cedarlane) which will bind to the Red Blood Cell/Rubella Antigen/Immunoglobulin complex at the level of the Fc fragment of the Immunoglobulin and cause the lysis of the red blood cells, is added to the preparation.

The absorbance at 541 nm of the mixture, which is a function of the haemolysis of the red blood cells, is measured using a (TECAN) SUNRISE spectrophotometer. The integrity of the Fc function is expressed by the ratio of the slope of the haemolysis curve of the sample to that of the reference considered to be 100%.

According to an advantageous embodiment, the present invention relates to a use in which said storage of said composition is such that, following said storage of said composition, the quantity of immunoglobulins that can bind to the RFcγ IIIa or CD16a receptor is equal to that of the composition before storage, plus or minus 20%.

One technology used can be Surface Plasmon Resonance (SPR). The interaction is then studied by carrying out different injections of immunoglobulins at different concentrations on a sensor chip prepared with CD16a covalently immobilized using an amine coupling kit (Biacore®, ref. BR-1000-50). The experimental conditions are specific to the Biacore T100. The data are interpreted by means of the Biacore T100 Evaluation software version 2.

According to an advantageous embodiment, the present invention relates to a use in which the syringe comprises:
a syringe barrel comprising an opening at each of its two ends, the first opening bearing means for sealing in a leakproof manner, in particular a nozzle cap, and/or means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
a composition, in particular pharmaceutical, comprising immunoglobulins,
   said syringe barrel, said sealing and/or administration means, and said sealing means capable of sliding delimiting a volume in which said composition is contained.

Said volume, in which said composition is contained, is less than or equal to the maximum volume that said syringe can contain. This first volume can be modified by causing said sealing means to slide inside the plunger barrel, the first opening being in particular free of sealing means.

When the first opening bears leakproof sealing means, in particular a nozzle cap and administration means, in particular a needle, said sealing means, initially leakproof, are subsequently pierced during the insertion of said administration means. In this case, it is not necessary to replace said sealing means by said administration means in order to administer said composition.

A plunger rod can be adapted to said plunger head, in order to allow the administration of said composition, said administration being manual, or carried out by automated administration means not comprising a plunger rod. An example of said automated administration means not comprising a plunger rod is the Niki T34L type pump (T60) “Ambulatory syringe pump” marketed by CME.

Alternatively, automated administration means comprising a plunger rod can directly allow said plunger head to slide in the direction of the first opening. An example of said automated administration means comprising a plunger rod is the Crono Super PID “syringe pump” marketed by Cane.

According to a particularly advantageous embodiment, the present invention relates to a use in which the syringe comprises:
a syringe barrel comprising an opening at each of its two ends, the first opening bearing means for sealing in a leakproof manner, in particular a nozzle cap, or the means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
a composition, in particular pharmaceutical, comprising immunoglobulins,
   said syringe barrel, said sealing or administration means, and said sealing means capable of sliding delimiting a volume in which said composition is contained.

According to an advantageous embodiment, the present invention relates to a use in which said storage of said composition is such that, following said storage of said composition, the leachables content in said composition, originating from the syringe barrel and/or the nozzle cap and/or the plunger head is acceptable for a pharmaceutical composition.

By leachable component is meant any component originating from the primary container (syringe barrel and/or nozzle cap and/or plunger head) or the secondary container (ink or label adhesive, blister) and which is transferred during storage into the composition. The leachables are dependent on the material used for the syringe barrel and/or the nozzle cap and/or the plunger head, and are also dependent on the additives used, for example for the coating of the syringe barrel.

The leachables can be in particular silicone oil, bisphenol, heavy metals, of bromo- or chlorobutyl, phthalate bromides (such as di-2-ethylhexyl phthalate or DEHP), volatile or non-volatile components.

According to an advantageous embodiment, the present invention relates to a use in which said storage of said composition is such that, following said storage of said composition:
the number of subvisible particles of a size greater than 10 μm is less than or equal to approximately 3000 per ml of composition, and/or
the number of subvisible particles of a size greater than 25 μm is less than or equal to approximately 300 per ml of composition, and/or
the dynamic light scattering measurement value of the composition administered is equal to that of the composition before storage, plus or minus 20%, and/or
the concentration of antibodies directed against the hepatitis B surface antigen is equal to that of the composition before storage, plus or minus 20%, and/or
there is integrity of the Fc function of at least 60% of said immunoglobulins, and/or
the quantity of immunoglobulins that can bind to the RFcγ IIIa or CD16a receptor is equal to that of the composition before storage, plus or minus 20%, and/or
the leachables content in said composition, originating from the syringe barrel and/or the nozzle cap and/or the plunger head is acceptable for a pharmaceutical composition.

According to an advantageous embodiment, the present invention relates to a use in which the duration of the storage is comprised from 0 months to 3 years.

According to an advantageous embodiment, the present invention relates to a use in which said syringe also comprises means for the automated administration of said composition, in particular pharmaceutical.

Said means for the automated administration of said composition are in particular pumps, more particularly portable pumps, for example the Niki T34L type pump (T60) “Ambulatory syringe pump” or the Crono Super PID “syringe pump”.

According to a particularly advantageous embodiment, the present invention relates to a use in which said administration means make it possible to administer said composition in an automated manner at a flow rate comprised from 5 to 50 ml/h, in particular from 20 to 40 ml/h.

According to another particularly advantageous embodiment, the present invention relates to a use in which said administration means make it possible to administer said composition in an automated manner at a flow rate comprised from 5 to 50 ml/h, in particular from 20 to 40 ml/h, said syringe being provided with a needle with a diameter comprised from gauge 29 to gauge 23, in particular from gauge 29 to gauge 27.

The invention also relates to a syringe pre-filled with a composition, in particular pharmaceutical, comprising immunoglobulins, allowing both the storage and the administration of said composition.

According to an advantageous embodiment, the present invention relates to a syringe having a permeability such that the variation in weight of the syringe containing said composition is at most 5%, compared with the initial weight of said syringe.

The invention also relates to an assembly constituted by a syringe and a composition, in particular pharmaceutical, comprising immunoglobulins, in particular polyvalent immunoglobulins, more particularly human immunoglobulins G,

said syringe being pre-filled with said composition, said syringe allowing both the storage and the administration of said composition,
said syringe also comprising a syringe barrel comprising an opening at each of its two ends, the first opening bearing means for sealing in a leakproof manner, in particular a nozzle cap, and/or means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
said syringe barrel, said sealing and/or administration means, and said sealing means capable of sliding delimiting a volume in which said composition is contained,
the viscosity of said composition, at 25° C. and under atmospheric pressure, being comprised from 2 to 200 mPa·s.

According to an advantageous embodiment, the present invention relates to an assembly in which said syringe has a permeability such that the variation in weight of the syringe containing said composition is at most 5%, compared with the initial weight of said syringe.

In particular, said syringe has a permeability such that the variation in weight of the syringe containing said composition is at most 4, 3, 2 or 1%, compared with the initial weight of said syringe.

More particularly, said syringe has a permeability such that after at least 12 months' storage, the variation in weight of the syringe containing said composition is at most 5% compared with the initial weight of said syringe.

More particularly, said syringe has a permeability such that after at least 12 months' storage, the variation in weight of the syringe containing said composition is at most 4, 3, 2 or 1%, compared with the initial weight of said syringe.

According to an advantageous embodiment, the present invention relates to an assembly or a syringe in which the molar percentage of immunoglobulins in monomer or dimer form is greater than 85%.

According to an advantageous embodiment, the present invention relates to an assembly or a syringe also comprising a material facilitating the movement of said sealing means capable of sliding, in particular of said plunger head, said material being in particular silicone, the leachables content, in particular silicone, in said composition being less than the quantities permissible for humans.

In the case of silicone, the permissible values are, for said composition contained in said syringe, for example at most 6000 particles the size of which is greater than or equal to 10 μm, and/or at most 600 particles the size of which is greater than or equal to 25 μm.

According to an advantageous embodiment, the present invention relates to an assembly or a syringe in which said immunoglobulins are polyvalent immunoglobulins.

According to an advantageous embodiment, the present invention relates to an assembly or a syringe in which the immunoglobulin concentration of said composition, in particular pharmaceutical, is comprised from 100 to 300 g/L.

According to a particularly advantageous embodiment, the present invention relates to an assembly or a syringe in which the immunoglobulin concentration of said composition, in particular pharmaceutical, is comprised from 200 to 300 g/L, preferably from 230 to 270 g/L.

According to another particularly advantageous embodiment, the present invention relates to an assembly or a syringe in which the immunoglobulin concentration of said composition, in particular pharmaceutical, is comprised from 100 to 200 g/L

According to an advantageous embodiment, the present invention relates to an assembly or a syringe in which the viscosity of said composition, in particular pharmaceutical, at 25° C. and under atmospheric pressure, is comprised from 2 to 200 mPa·s.

According to a particularly advantageous embodiment, the present invention relates to an assembly or a syringe in which the viscosity of said composition, in particular pharmaceutical, at 25° C. and under atmospheric pressure, is comprised from 12 to 200 mPa·s, preferably from 20 to 80 mPa·s.

According to another particularly advantageous embodiment, the present invention relates to an assembly or a syringe in which the viscosity of said composition, in particular pharmaceutical, at 25° C. and under atmospheric pressure, is comprised from 2 to 12 mPa·s.

According to an advantageous embodiment the present invention relates to an assembly or a syringe in which the volume of said composition, in particular pharmaceutical, stored in said syringe is comprised from 1 to 50 ml, preferably from 2 to 35 ml, more preferentially from 4 to 20 ml.

Said volume of said composition is less than or equal to the maximum volume that said syringe can contain.

The present invention relates to an assembly or a syringe in which the volume of said composition, in particular pharmaceutical, stored in said syringe is comprised from 1 to 10 ml, preferably from 2 to 10 ml, more preferentially from 4 to 10 ml.

According to another particularly advantageous embodiment, the present invention relates to an assembly or a syringe in which the volume of said composition, in particular pharmaceutical, stored in said syringe is comprised from 10 to 50 ml, preferably from 10 to 35 ml, more preferentially from 10 to 20 ml.

According to an advantageous embodiment, the present invention relates to an assembly or a syringe comprising means, in particular a needle, for the administration of said composition, in particular pharmaceutical, by intradermal, intramuscular, intravenous and sub-cutaneous, in particular sub-cutaneous, route.

According to a particularly advantageous embodiment, the present invention relates to an assembly or a syringe in which said needle has a diameter comprised from gauge 29 to gauge 23, in particular from gauge 29 to gauge 27.

According to an advantageous embodiment, the present invention relates to an assembly or a syringe in which the volume of said composition, in particular pharmaceutical, stored in said syringe is comprised from 1 to 50 ml, preferably from 2 to 35 ml, more preferentially from 4 to 20 ml,

said syringe comprising means, in particular a needle, for the administration of said composition, in particular pharmaceutical, by intradermal, intramuscular, intravenous and sub-cutaneous, in particular sub-cutaneous, route.

According to an advantageous embodiment, the present invention relates to an assembly or a syringe in which:
the volume of said composition, in particular pharmaceutical, stored in said syringe is comprised from 1 to 50 ml, preferably from 2 to 35 ml, more preferentially from 4 to 20 ml,
the immunoglobulin concentration of said composition, in particular pharmaceutical, is comprised from 100 to 300 g/L, preferably from 200 to 300 g/L, more preferentially from 230 to 270 g/L,
   said syringe comprising means, in particular a needle, for the administration of said composition, in particular pharmaceutical, by intradermal, intramuscular, intravenous and sub-cutaneous, in particular sub-cutaneous, route.

According to an advantageous embodiment, the present invention relates to an assembly or a syringe in which said immunoglobulins are polyvalent, said polyvalent immunoglobulins being in particular human immunoglobulins G.

According to an advantageous embodiment, the present invention relates to an assembly or a syringe in which said composition is in the form of an aqueous solution.

According to an advantageous embodiment, the present invention relates to an assembly or a syringe in which said composition, in particular pharmaceutical, comprises:
human polyvalent immunoglobulins at a concentration of from 100 to 300 g/L, preferably from 200 to 300 g/L, more preferentially from 230 to 270 g/L, even more preferentially of approximately 250 g/L,
one or more hydrophobic amino acids, preferably glycine,
optionally a buffer, preferably sodium acetate buffer,
a non-ionic surfactant, preferably polysorbate, more preferentially polysorbate 80, or a poloxamer.

According to a particularly advantageous embodiment, the present invention relates to an assembly or a syringe in which said composition, in particular pharmaceutical, comprises:
human polyvalent immunoglobulins at a concentration of from 100 to 300 g/L, preferably from 200 to 300 g/L, more preferentially from 230 to 270 g/L, even more preferentially of approximately 250 g/L,
glycine,
sodium acetate buffer,
polysorbate, preferably polysorbate 80, or a poloxamer.

According to an advantageous embodiment, the present invention relates to an assembly or a syringe in which said storage of said composition is such that, following said storage of said composition, the number of subvisible particles of a size greater than 10 μm is less than or equal to approximately 3000 per ml of composition.

According to another advantageous embodiment, the present invention relates to an assembly or a syringe in which said storage of said composition is such that, following said storage of said composition, the number of subvisible particles of a size greater than 25 μm is less than or equal to approximately 300 per ml of composition.

According to an advantageous embodiment, the present invention relates to an assembly or a syringe in which said storage of said composition is such that, following said storage of said composition, the dynamic light scattering measurement value of the composition administered is equal to that of the composition before storage, plus or minus 20%.

According to an advantageous embodiment, the present invention relates to an assembly or a syringe in which said storage of said composition is such that, following said storage of said composition, the concentration of antibodies directed against the hepatitis B surface antigen is equal to that of the composition before storage, plus or minus 20%.

According to an advantageous embodiment, the present invention relates to an assembly or a syringe in which said storage of said composition is such that, following said storage of said composition, there is integrity of the Fc function of at least 60% of said immunoglobulins.

According to an advantageous embodiment, the present invention relates to an assembly or a syringe in which said storage of said composition is such that, following said storage of said composition, the quantity of immunoglobulins that can bind to the RFcγ IIIa or CD16a receptor is equal to that of the composition before storage, plus or minus 20%.

According to an advantageous embodiment, the present invention relates to a syringe comprising:
a syringe barrel comprising an opening at each of its two ends, the first opening bearing means for sealing in a leakproof manner, in particular a nozzle cap, and/or means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
a composition, in particular pharmaceutical, comprising immunoglobulins,
   said syringe barrel, said sealing and/or administration means, and said sealing means capable of sliding delimiting a volume in which said composition is contained.

Said volume, in which said composition is contained, is less than or equal to the maximum volume that said syringe can contain. This first volume can be modified by causing said sealing means to slide inside the plunger barrel, the first opening being in particular free of sealing means.

When the first opening bears leakproof sealing means, in particular a nozzle cap and administration means, in particular a needle, said sealing means, initially leakproof, are subsequently pierced during the insertion of said administration means. In this case, it is not necessary to replace said sealing means by said administration means in order to administer said composition.

A plunger rod can be adapted to said plunger head, in order to allow the administration of said composition, said administration being manual, or carried out by automated administration means not comprising a plunger rod. An example of said automated administration means not comprising a plunger rod is the Niki T34L type pump (T60) “Ambulatory syringe pump”.

Alternatively, automated administration means comprising a plunger rod, can directly allow said plunger head to slide in the direction of the first opening. An example of said automated administration means comprising a plunger rod is the Crono Super PID “syringe pump”.

According to a particularly advantageous embodiment, the present invention relates to an assembly or a syringe comprising:
a syringe barrel comprising an opening at each of its two ends, the first opening bearing means for sealing in a leakproof manner, in particular a nozzle cap, or means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
a composition, in particular pharmaceutical, comprising immunoglobulins,
   said syringe barrel, said sealing or administration means, and said sealing means capable of sliding delimiting a volume in which said composition is contained.

According to an advantageous embodiment, the present invention relates to an assembly or a syringe in which said first opening is sealed with a nozzle cap.

According to an advantageous embodiment, the present invention relates to an assembly or a syringe in which said first opening is equipped with a needle, in particular a needle intended for sub-cutaneous administration.

According to a particularly advantageous embodiment, the present invention relates to an assembly or a syringe in which said needle has a diameter comprised from gauge 29 to gauge 23, in particular from gauge 29 to gauge 27.

According to an advantageous embodiment, the present invention relates to an assembly or a syringe in which said syringe barrel is constituted by, or comprises, a material with a low leachables content.

By “low leachables content”, is meant a leachables content in said composition, being less than the quantities permissible for humans.

According to a particularly advantageous embodiment, the present invention relates to an assembly or a syringe in which said syringe barrel is constituted by, or comprises, a material with a low leachables content chosen from glass, in particular glass of type 1 and glass of type 1+, cyclo-olefin copolymers, cyclo-olefin polymers, and polypropylene.

According to an advantageous embodiment, the present invention relates to an assembly or a syringe in which said nozzle cap is constituted by, or comprises, a material with a low leachables content.

According to a particularly advantageous embodiment, the present invention relates to an assembly or a syringe in which said nozzle cap is constituted by, or comprises, a material with a low leachables content chosen from the chlorobutyl, bromobutyl, and bromobutyl-polyisoprene elastomers.

According to an advantageous embodiment, the present invention relates to an assembly or a syringe in which said plunger head is constituted by, or comprises, a material with a low leachables content.

According to a particularly advantageous embodiment, the present invention relates to an assembly or a syringe in which said plunger head is constituted by, or comprises, a material with a low leachables content chosen from the chlorobutyl, bromobutyl, and styrene-butadiene elastomers.

According to an advantageous embodiment, the present invention relates to an assembly or a syringe in which said storage of said composition is such that, following said storage of said composition, the leachables content in said composition, originating from the syringe barrel and/or the nozzle cap and/or the plunger head is acceptable for a pharmaceutical composition.

According to an advantageous embodiment, the present invention relates to an assembly or a syringe in which said storage of said composition is such that, following said storage of said composition:
the number of subvisible particles of a size greater than 10 μm is less than or equal to approximately 3000 per ml of composition, and/or
the number of subvisible particles of a size greater than 25 μm is less than or equal to approximately 300 per ml of composition, and/or
the dynamic light scattering measurement value of the composition administered is equal to that of the composition before storage, plus or minus 20%, and/or
the concentration of antibodies directed against the hepatitis B surface antigen is equal to that of the composition before storage, plus or minus 20%, and/or
there is integrity of the Fc function of at least 60% of said immunoglobulins, and/or
the quantity of immunoglobulins that can bind to the RFcγ IIIa or CD16a receptor is equal to that of the composition before storage, plus or minus 20%, and/or
the leachables content in said composition, originating from the syringe barrel and/or the nozzle cap and/or the plunger head is acceptable for a pharmaceutical product.

According to an advantageous embodiment, the present invention relates to an assembly or a syringe in which:
said syringe barrel is constituted by, or comprises a cyclo-olefin polymer,
said nozzle cap is constituted by, or comprises a chlorobutyl elastomer,
said plunger head is constituted by, or comprises, a chlorobutyl elastomer.

According to another advantageous embodiment, the present invention relates to an assembly or a syringe in which:
said syringe barrel is constituted by, or comprises a cyclo-olefin polymer,
said nozzle cap is constituted by, or comprises a cyclo-olefin polymer,
said plunger head is constituted by, or comprises a styrene-butadiene elastomer.

According to yet another advantageous embodiment the present invention relates to an assembly or a syringe in which:
said syringe barrel is constituted by, or comprises glass,
said nozzle cap is constituted by, or comprises a bromobutyl-polyisoprene elastomer,
said plunger head is constituted by, or comprises, a chlorobutyl elastomer.

According to yet another advantageous embodiment, the present invention relates to an assembly or a syringe in which:
said syringe barrel is constituted by, or comprises a cyclo-olefin copolymer,
said nozzle cap is constituted by, or comprises a bromobutyl elastomer,
said plunger head is constituted by, or comprises, a chlorobutyl elastomer.

The invention also relates to a kit comprising a pre-filled syringe as described previously and hermetic packaging containing said syringe, said packaging being in particular made of aluminium.

The invention also relates to a kit comprising an assembly as described previously and hermetic packaging containing said syringe, said packaging being in particular made of aluminium.

The invention also relates to a kit comprising a pre-filled syringe as described previously, allowing both the storage and the administration of a composition, in particular pharmaceutical, comprising immunoglobulins, in particular polyvalent immunoglobulins, and means for the automated administration of said composition, in particular pharmaceutical.

The invention also relates to a kit comprising an assembly constituted by a syringe and a composition, and means for the automated administration of said composition, in particular pharmaceutical.

Said means for the automated administration of said composition are in particular pumps, more particularly portable pumps, for example the Niki T34L type pump (T60) “Ambulatory syringe pump” or the Crono Super PID “syringe pump”.

According to an advantageous embodiment, the present invention relates to a kit in which said administration means make it possible to administer said composition in an automated manner at a flow rate comprised from 5 to 50 ml/h, in particular from 20 to 40 ml/h.

According to a particularly advantageous embodiment, the present invention relates to a kit in which said administration means make it possible to administer said composition in an automated manner at a flow rate comprised between 5 and 50 ml/h, in particular between 20 and 40 ml/h, said syringe being provided with a needle with a diameter comprised from gauge 29 to gauge 23, in particular from gauge 29 to gauge 27.

The invention also relates to a method for the preparation of a pre-filled syringe as described previously, comprising:
a composition, in particular pharmaceutical, comprising immunoglobulins,
a syringe barrel comprising an opening at each of its two ends, the first opening being sealed in a leakproof manner by sealing means, in particular a nozzle cap, the second opening being sealed in a leakproof manner by sealing means, in particular a plunger head, capable of sliding inside the plunger barrel, in particular using a plunger rod linked to said sealing means,
   said syringe barrel, said sealing means and said sealing means capable of sliding delimiting a volume in which said composition is contained,
   said method comprising the following stages:
a) filling of a syringe barrel comprising an opening at each of its two ends, the first opening being sealed in a leakproof manner by sealing means, in particular a nozzle cap, with a composition, in particular pharmaceutical, comprising immunoglobulins, said filling being carried out via the second opening, in order to obtain a syringe barrel containing said composition;
b) sealing in a leakproof manner of the second opening of the syringe barrel obtained in stage a) by sealing means, in particular a plunger head, capable of sliding inside the plunger barrel, in order to obtain said pre-filled syringe.

The invention also relates to a method for the preparation of a pre-filled syringe as described previously, comprising:
a composition, in particular pharmaceutical, comprising immunoglobulins,
a syringe barrel comprising an opening at each of its two ends, the first opening being sealed in a leakproof manner by sealing means, in particular a nozzle cap, the second opening being sealed in a leakproof manner by sealing means, in particular a plunger head, capable of sliding inside the plunger barrel, in particular using a plunger rod linked to said sealing means,
   said syringe barrel, said sealing means, and said sealing means capable of sliding delimiting a volume in which said composition is contained,
   said method comprising the following stages:
a) filling of a syringe barrel comprising an opening at each of its two ends, the second opening being sealed in a leakproof manner by sealing means, in particular a plunger head, capable of sliding inside the plunger barrel, with a composition, in particular pharmaceutical, comprising immunoglobulins, said filling being carried out via the first opening, in order to obtain a syringe barrel containing said composition;
b) sealing in a leakproof manner of the first opening of the syringe barrel obtained in stage a) by sealing means, in particular a nozzle cap, in order to obtain said pre-filled syringe.

The invention also relates to a method for the preparation of an assembly as described previously, comprising:
a composition, in particular pharmaceutical, comprising immunoglobulins,
a syringe barrel comprising an opening at each of its two ends, the first opening being sealed in a leakproof manner by sealing means, in particular a nozzle cap, the second opening being sealed in a leakproof manner by sealing means, in particular a plunger head, capable of sliding inside the plunger barrel, in particular using a plunger rod linked to said sealing means,
   said syringe barrel, said sealing means, and said sealing means capable of sliding delimiting a volume in which said composition is contained,
   said method comprising the following stages:
c) filling of a syringe barrel comprising an opening at each of its two ends, the first opening being sealed in a leakproof manner by sealing means, in particular a nozzle cap, with a composition, in particular pharmaceutical, comprising immunoglobulins, said filling being carried out via the second opening, in order to obtain a syringe barrel containing said composition;
d) sealing in a leakproof manner of the second opening of the syringe barrel obtained in stage a) by sealing means, in particular a plunger head, capable of sliding inside the plunger barrel, in order to obtain said pre-filled syringe.

The invention also relates to a method for the preparation of an assembly as described previously, comprising:
a composition, in particular pharmaceutical, comprising immunoglobulins,
a syringe barrel comprising an opening at each of its two ends, the first opening being sealed in a leakproof manner by sealing means, in particular a nozzle cap, the second opening being sealed in a leakproof manner by sealing means, in particular a plunger head, capable of sliding inside the plunger barrel, in particular using a plunger rod linked to said sealing means,
   said syringe barrel, said sealing means, and said sealing means capable of sliding delimiting a volume in which said composition is contained,
   said method comprising the following stages:
c) filling of a syringe barrel comprising an opening at each of its two ends, the second opening being sealed in a leakproof manner by sealing means, in particular a plunger head, capable of sliding inside the plunger barrel, with a composition, in particular pharmaceutical, comprising immunoglobulins, said filling being carried out via the first opening, in order to obtain a syringe barrel containing said composition;
sealing in a leakproof manner, of the first opening of the syringe barrel obtained in stage a) by sealing means, in particular a nozzle cap, in order to obtain said pre-filled syringe.

According to an advantageous embodiment, the present invention relates to a method in which said volume comprising said composition is substantially gas-free.

According to another advantageous embodiment, the present invention relates to a method in which said volume containing said composition also contains an inert gas, in particular nitrogen.

### DESCRIPTION OF THE FIGURES

FIG. 1 shows an example curve obtained for determining the viscosity of an immunoglobulin solution at 249 g/L.

FIG. 2 represents a pre-fillable syringe, intended to be filled with an immunoglobulin composition, to form a pre-filled syringe containing said immunoglobulin composition.

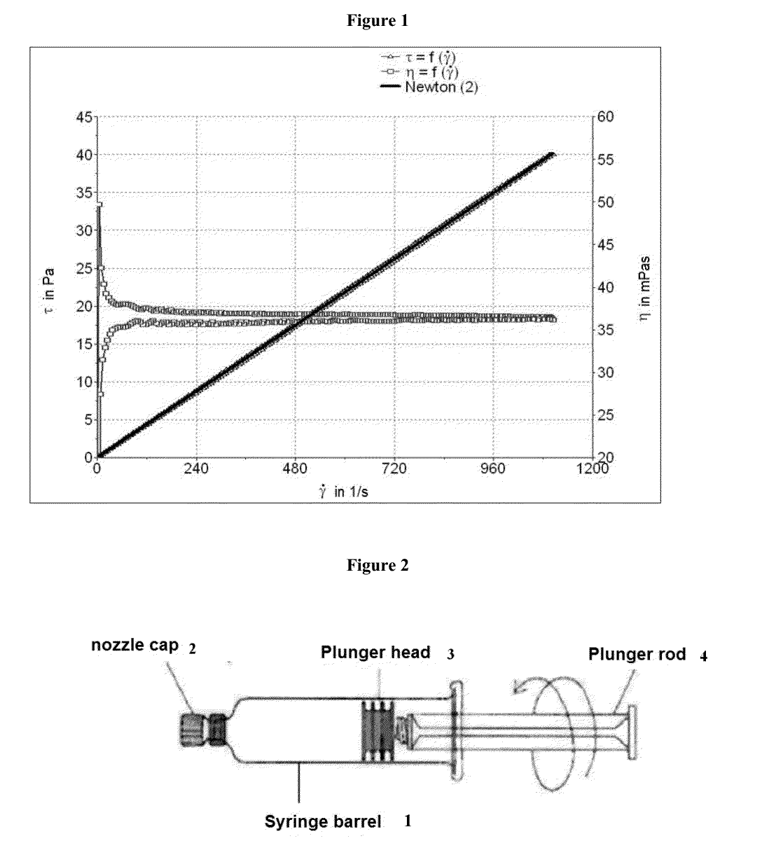

This pre-fillable syringe comprises a syringe barrel (1), a nozzle cap (2), a plunger head (3) and optionally a plunger rod (4).

The following Examples 1 to 5 illustrate the invention.

### EXAMPLES

### Example 1

### Pre-Filled Syringe C1

A syringe the characteristics of which are noted in Table 2 is filled to 4 ml with a solution of immunoglobulins the formulation of which is as follows:
IGNG 251 g/L;
Acetate buffer: 40 mM;
Glycine: 187 mM;
Polysorbate 80: 200 ppm.

The pH of this solution is 4.8.

The viscosity measured at 25° C. and at atmospheric pressure of this solution is 43.25 mPa·s.

TABLE 2
composition of the syringe allowing the pre-
filled syringe C1 to be obtained after filling.

| Compo- | | Manufacturer's | Materials/ |
|---|---|---|---|
| nents | Name | reference | formulation |
| Syringe | Syringe 10 mL | Barrel 10LL-3 | Plastic: Daikyo |
| barrel | Crystal Zenith | | Resin CZ |
| Nozzle cap | LL Nozzle Cap NF | LL Nozzle Cap NF | Chlorobutyl |
| | | | D21-6-1 |
| Plunger head | WPS/Daikyo | 10 mL Piston | Chlorobutyl |
| | D21-6-1 | FR2-N1 | D21-6-1 |
| Plunger rod | Plunger 10 PP | Plunger 10 | polypropylene |

### Example 2

### Pre-Filled Syringe C2

A syringe the characteristics of which are noted in Table 3 is filled to 4 ml with an immunoglobulin solution the formulation of which is as follows:
IGNG 251 g/L;
Acetate buffer: 40 mM;
Glycine: 187 mM;
Polysorbate 80: 200 ppm.

The pH of this solution is 4.8.

The viscosity measured at 25° C. and at atmospheric pressure of this solution is 43.25 mPa·s.

TABLE 3
composition of the syringe allowing the pre-
filled syringe C2 to be obtained after filling.

| Compo- | | Manufacturer's | Materials/ |
|---|---|---|---|
| nents | Name | reference | formulation |
| Syringe | BD STERIFILL ™ | 47233001 | Plastic: |
| barrel | 10 ML CCP with | | Crystal Clear |
| | Tip Cap | | Polymer resin |
| Nozzle cap | NA, provided with | NA, provided | CCP resin + |
| | syringe barrel | with syringe | thermoplastic |
| | | barrel | elastomer |
| Plunger head | BD STERIFILL ™ | 47236919 | Styrene- |
| | NSCF10 ML | | Butadiene |
| | SG020J02BLACK | | Rubber (SBR) |
| | SI1000 | | |
| Plunger rod | 10401PR PPYL | 47103508 | polypropylene |
| | Natural | | |

### Example 3

### Pre-Filled Syringe C3

A syringe the characteristics of which are noted in Table 4 is filled to 4 ml with an immunoglobulin solution the formulation of which is as follows:
IGNG 251 g/L;
Acetate buffer: 40 mM;
Glycine: 187 mM;
Polysorbate 80: 200 ppm.

The pH of this solution is 4.8.

The viscosity measured at 25° C. and at atmospheric pressure of this solution is 43.25 mPa·s.

TABLE 4
composition of the syringe allowing the pre-
filled syringe C3 to be obtained after filling.

| Compo- | | Manufacturer's | Materials/ |
|---|---|---|---|
| nents | Name | reference | formulation |
| Syringe | BD Hypak ™ | 47179819 | Glass |
| barrel | SCF10ML LL3 | | |
| Nozzle cap | PRTC7025/65GR | NA provided with | Synthetic isoprene + |
| | | syringe barrel | Bromobutyl |
| Plunger head | Piston 10 mL | 6901GCC3720 | Chlorobutyl |
| | Stelmi C3720 | | 6901GC |
| Plunger rod | 10401PR PPYL | 47103508 | polypropylene |
| | Natural | | |

### Example 4

### Pre-Filled Syringe C4

A syringe the characteristics of which are noted in Table 5 is filled to 4 ml with an immunoglobulin solution the formulation of which is as follows:
IGNG 251 g/L;
Acetate buffer: 40 mM;
Glycine: 187 mM;
Polysorbate 80: 200 ppm.

The pH of this solution is 4.8.

The viscosity measured at 25° C. and at atmospheric pressure of this solution is 43.25 mPa·s.

TABLE 5
composition of the syringe allowing the pre-
filled syringe C4 to be obtained after filling.

| Compo- | | Manufacturer's | Materials/ |
|---|---|---|---|
| nents | Name | reference | formulation |
| Syringe | SCHOTT | 3154A | Plastic: |
| barrel | TopPac ® | | TOPAS ®, ad- |
| | 10 mL cut | | vanced cyclic |
| | finger flange | | olefin copolymer |
| Nozzle cap | Helvoet FM257/2 | NA, provided with | Bromobutyl |
| | | syringe barrel | FM257/2 |
| | | | dark grey |
| Plunger head | Piston 10 mL | 6901GCC3720 | Chlorobutyl |
| | Stelmi C3720 | | 6901GC |
| Plunger rod | SCHOTT | NA, provided with | polypropylene |
| | TopPac ® plung- | syringe barrel | |
| | er rod 10 mL | | |

### Example 5

### Stability of the Pre-Filled Syringes

### Example 5.1

### Stability Testing

The storage conditions for the syringes C1-C4 are indicated in Table 6.

TABLE 6
storage conditions relating to the syringes C1-C4

| Packaging | Storage conditions |
|---|---|
| Syringe C3 | 5° C. ± 3° C., |
| | 25° C. ± 2° C./60% RH ± 5% RH |
| | 40° C. ± 2° C./75% RH ± 5% RH |
| Syringes C1, C2 and C4 | 5° C. ± 3° C., |
| | 25° C. ± 2° C./40% RH ± 5% RH |
| | 40° C. ± 2° C./≦25% RH |

### Example 5.2

### Analyses Carried Out for the Stability Study

In addition to the evaluation of the appearance of the solution, measurements of the turbidity at 350 nm, the optical density at 280 nm, the pH, the osmolality, the ADCC (for antibody-dependant cell-mediated cytotoxicity), the MSD (molecular size distribution), the polysorbate 80 concentration, the extractible volume, the mass and the microbial contamination, the following analyses were carried out:
non-visible particles less than 50 μm: subvisible particles, greater than 10 μm and greater than 25 μm were counted by flow microscopy (MFI) on a total volume of 2 mL;
   by subvisible particles, is meant particles less than 50 μm but greater than 2 μm, greater than 10 μm and greater than 25 μm which are counted by flow microscopy (MFI) on a total volume of 2 mL. This analysis makes it possible to quantify the content of subvisible particles in addition to visual inspection;
   the analysis is carried out on a BrightWELL/DPA4100 Flow Microscope in LowMag (HM) configuration (magnification ×5). The data collected are displayed and can be processed using the MFI View software;
dynamic light scattering (DLS) measurement, allowing measurement of the sizes (hydrodynamic radius) of the objects in solution, between approximately 1 nm and 1 μm;
   the ALV/CGS COMPACT GONIOMETER SYSTEM light scattering platform is used for the dynamic light scattering (DLS) measurements and allows determination of the hydrodynamic radius HR of colloidal solutions, mainly of proteins, corresponding to the scattering objects which are less than approximately 1 μm; in order to manage the light scattering platform a computer with ALV/CGS3 software is connected to the ALV/LES-5004 panel. The wavelength used for the analysis is λ=632.80 nm;
assay of the antibodies directed against the hepatitis B surface (anti-Hbs) antigen:
   the quantitative determination of the anti-hepatitis B activity of the immunoglobulins is carried out by the ELISA immunoenzymatic technique; the tests are carried out using an ETI-AB-AUK-3 Anti-HBs EIA kit (CE marked), manufactured and marketed by DIASORIN; the titration corresponds to the parallel line assay model described in the European Pharmacopoeia; this titration is carried out on the ETI-MAX 3000 microplate manager (DIASORIN);
measurement of the integrity of the Fc function: this analysis makes it possible to monitor the integrity of the Fc function of the Immunoglobulins, i.e. their complement-binding ability; the rubella antigen (Aalto) is bound to human red blood cells (originating from human blood of group O) then brought into contact with the Immunoglobulin preparations to be tested; after a time necessary for the formation of immune complexes, guinea pig complement (Tebu-Bio Cedarlane) which will bind to the Red Blood Cell/Rubella Antigen/Immunoglobulin complex at the level of the Fc fragment of the Immunoglobulin and will cause the lysis of the red blood cells is added to the preparation;
   the absorbance at 541 nm of the mixture, which is a function of the haemolysis of the red blood cells is measured using a (TECAN) SUNRISE spectrophotometer. The integrity of the Fc function is expressed by the ratio of the slope of the hemolysis curve of the sample to that of the reference considered as 100%;
CD16 binding: this analysis makes it possible to monitor the integrity of the protein by monitoring the interaction with the RFcγ IIIa or CD16a receptor;
   the technology used is Surface Plasmonic Resonance (SPR). The interaction is studied by carrying out different injections of immunoglobulins at different concentrations on a sensor chip prepared with CD16a covalently immobilized using an amine coupling kit (Biacore®, ref. BR-1000-50); the experimental conditions are specific to Biacore T100. The data are interpreted using the Biacore T100 Evaluation software version 2;
measurement of the leachables content.

### Example 5.3

### Stability at One Month

The stability of the syringes stored at 25° C. and at 40° C. was evaluated after one month.

The values obtained for the syringes stored at 25° C. and at 40° C. are as follows:
appearance of the solution: clear, slightly yellow, particle-free;
subvisible particles: particles >10 μm: <3000/ml; particles >25 μm: <300/ml;
DLS: value at T0+/−20%;
Turbidity at 350 nm: 0.350+/−10%;
OD 280 nm: 251 g/L
pH: 4.8;
osmolality: value at T0+/−20%;
CD16 interaction: value at T0+/−20%;
MSD: monomer+dimer> or equal to 85%; polymer: 3-4%; fragment: 1.5%; the molecular size distribution is determined by gel filtration chromatography (High Performance Size Exclusion Chromatography=HPSEC) on a superose 12 type prepacked chromatography column, 10/300 GL TRICORN from GE Healthcare, using for example a phosphate buffer adjusted to pH 6.7; it consists of a separation of the proteins of molecular mass comprised between 10³ and 3.10⁵ Daltons on a superose gel;
Anti HBs: value at T0+/−20%;
mass: value at T0+/−5%.

The values measured on the syringes C1-C4 stored for one month at 25 and 40° C. were similar to those observed for an immunoglobulin solution of the same composition, stored under the same conditions of temperature (25 and 40° C. respectively) and of hygrometry as the syringe C3, in a SCHOTT DURAN glass bottle (bottle with a threaded neck (GL45 thread) without a screw cap, of 500 ml capacity, reference VWR 215-1536, sealed with a screw cap (GL45 thread) made of red polyester (PBT) with a PTFE coated seal, reference VWR 201-0004).

### Example 5.3

### Stability at 1, 3, 6 and 12 Months

The stability of the syringes stored at 5° C. was evaluated at 3, 6 and 12 months.

The stability of the syringes stored at 25° C. was evaluated at 1, 3, 6 and 12 months.

The stability of the syringes stored at 40° C. was evaluated at 1, 3 and 6 months.

The results relating to the syringes C1-4 are noted in the tables hereafter:

TABLE 7
stability of the syringe C1 at 5° C.

| | STABILITY 5° C. | Time period (months) | | | | |
|---|---|---|---|---|---|---|
| | Expected values | | T0 | T3M | T6M | T12M |
| Appearance of solution | particle-free (FO) | | Particle-free | Particle-free | Particle-free | Particle-free |
| MFI | total number of particles/mL | | 1516 | 2250 | 1378 | 1957 |
| | number of particles ≧10 μm/mL (max 6000 p/vial) | | 153 | 218 | 203 | 235 |
| | number of particles ≧25 μm/mL (max 600 p/vial) | | 12 | 26 | 28 | 60 |
| | number of silicone drops ≧10 μm/mL | | 0 | 0 | 0 | 0 |
| | number of silicone drops ≧25 μm/mL | | 0 | 0 | 0 | 0 |
| DLS (% of monomer) | at 30° in % | No change/T0 (20%) | 90.6 | 82.6 | 84.6 | 82.2 |
| | at 90° in % | No change/T0 (20%) | 100.0 | 98.6 | 94.2 | 96.6 |
| | at 150° in % | No change/T0 (20%) | 95.2 | 97.8 | 94.5 | 97.5 |
| | Id at 30° | No change/T0 (20%) | 68.55 | 71.06 | 70.60 | 65.54 |
| | Id at 90° | No change/T0 (20%) | 30.47 | 32.11 | 32.63 | 29.96 |
| Concentration | OD 280 nm | 225-275 g/L | 244.4 | 241.0 | 245.8 | 241.4 |
| CD16 binding | No change/T0 (20%) | | 111.0 | 122 | 117 | 117 |
| MSD | % of monomer | mono + dimers ≧ 85% | 90.2 | 90.5 | 89.5 | 89.6 |
| | % of dimer | | 8.5 | 8.4 | 9.3 | 9.4 |
| | % of polymer | ≦10% | <0.4 | <0.4 | 0.4 | 0.4 |
| | % of fragment | ≦3% | 1.1 | <1.0 | <1.0 | <1.0 |

| | | | | |
|---|---|---|---|---|
| Fc function | ≧60% | 120 | 99 | 104 |
| Microbial contamination | ≦10 CFU/100 ml | 0 CFU/8 mL | | |

TABLE 8
stability of the syringe C1 at 25° C.

| | STABILITY 25° C. | Time period (months) | | | | | |
|---|---|---|---|---|---|---|---|
| | Expected values | | T0 | T1M | T3M | T6M | T12M |
| Appearance of solution | particle-free (FO) | | Particle-free | Particle-free | Particle-free | Particle-free | Particle-free |
| MFI | total number of particles/mL | | 1516 | 2030 | 740 | 2063 | 9505 |
| | number of particles ≧10 μm/mL (max 6000 p/vial) | | 153 | 196 | 192 | 196 | 394 |
| | number of particles ≧25 μm/mL (max 600 p/vial) | | 12 | 28 | 30 | 21 | 32 |
| | number of silicone drops ≧10 μm/mL | | 0 | 0 | 0 | 0 | 0 |
| | number of silicone drops ≧25 μm/mL | | 0 | 0 | 0 | 0 | 0 |
| DLS (% of monomer) | at 30° in % | No change/T0 (20%) | 90.6 | 89.1 | 87.8 | 81.6 | 80.8 |
| | at 90° in % | No change/T0 (20%) | 100.0 | 94.7 | 96.2 | 89.2 | 83.3 |
| | at 150° in % | No change/T0 (20%) | 95.2 | 93.0 | 95.4 | 93.6 | 86.5 |
| | Id at 30° | No change/T0 (20%) | 68.55 | 66.85 | 78.59 | 78.31 | 76.87 |
| | Id at 90° | No change/T0 (20%) | 30.47 | 30.14 | 33.43 | 34.30 | 32.37 |
| Concentration | OD 280 nm | 225-275 g/L | 244.4 | 244.0 | 241.2 | 245.0 | 243.7 |
| CD16 binding | No change/T0 (20%) | | 111.0 | 108 | 110 | 98 | 100 |
| MSD | % of monomer | mono + dimers ≧ 85% | 90.2 | 91.1 | 90.6 | 88.8 | 88.0 |
| | % of dimer | | 8.5 | 7.5 | 8.0 | 9.4 | 9.7 |
| | % of polymer | ≦10% | <0.4 | <0.4 | <0.4 | 0.4 | 0.7 |
| | % of fragment | ≦3% | 1.1 | 1.1 | 1.1 | 1.3 | 1.6 |

| | | | | |
|---|---|---|---|---|
| Fc function | ≧60% | 120 | 92 | 100 |
| Microbial contamination | ≦10 CFU/100 ml | 0 CFU/8 mL | | |

TABLE 9
stability of the syringe C1 at 40° C.

| | STABILITY 40° C. | Time period (months) | | | | |
|---|---|---|---|---|---|---|
| | Expected values | | T0 | T1M | T3M | T6M |
| Appearance of solution | particle-free (FO) | | Particle-free | Particle-free | Particle-free | Particle-free |
| MFI | total number of particles/mL | | 1516 | 2763 | 941 | 2578 |
| | number of particles ≧10 μm/mL (max 6000 p/vial) | | 153 | 227 | 209 | 188 |
| | number of particles ≧25 μm/mL (max 600 p/vial) | | 12 | 38 | 26 | 20 |
| | number of silicone drops ≧10 μm/mL | | 0 | 0 | 0 | 0 |
| | number of silicone drops ≧25 μm/mL | | 0 | 0 | 0 | 0 |
| DLS (% of monomer) | at 30° in % | No change/T0 (20%) | 90.6 | 90.4 | NA | 8.5 |
| | at 90° in % | No change/T0 (20%) | 100.0 | 63.7 | 29.9 | 12.7 |
| | at 150° in % | No change/T0 (20%) | 95.2 | 64.4 | 27.6 | 12.2 |
| | Id at 30° | No change/T0 (20%) | 68.55 | 99.53 | 236.58 | 518.69 |
| | Id at 90° | No change/T0 (20%) | 30.47 | 45.35 | 110.90 | 253.60 |
| Concentration | OD 280 nm | 225-275 g/L | 244.4 | 242.6 | 242.7 | 252.9 |
| CD16 binding | No change/T0 (20%) | | 111.0 | 107 | 77 | 51 |
| MSD | % of monomer | mono + dimers ≧ 85% | 90.2 | 88.3 | 79.3 | 68.2 |
| | % of dimer | | 8.5 | 6.4 | 6.4 | 6.5 |
| | % of polymer | ≦10% | <0.4 | 3.8 | 12.0 | 21.8 |
| | % of fragment | ≦3% | 1.1 | 1.5 | 2.4 | 3.5 |

| | | | | |
|---|---|---|---|---|
| Fc function | ≧60% | 120 | 108 | 95 |
| Microbial contamination | ≦10 CFU/100 ml | 0 CFU/8 mL | | 0 CFU/8 mL |

TABLE 10
stability of the syringe C2 at 5° C.

| | STABILITY 5° C. | Time period (months) | | | | |
|---|---|---|---|---|---|---|
| | Expected values | | T0 | T3M | T6M | T12M |
| Appearance of solution | particle-free (FO) | | Particle-free | Particle-free | Particle-free | Particle-free |
| MFI | total number of particles/mL | | 5627 | 737 | 2495 | 27241 |
| | number of particles ≧10 μm/mL (max 6000 p/vial) | | 179 | 93 | 91 | 2202 |
| | number of particles ≧25 μm/mL (max 600 p/vial) | | 26 | 9 | 10 | 122 |
| | number of silicone drops ≧10 μm/mL | | 265 | 209 | 183 | 362 |
| | number of silicone drops ≧25 μm/mL | | 7 | 11 | 53 | 26 |
| DLS (% of monomer) | at 30° in % | No change/T0 (20%) | 92.0 | 90.0 | 87.1 | 87.9 |
| | at 90° in % | No change/T0 (20%) | 98.1 | 97.5 | 96.0 | 100.0 |
| | at 150° in % | No change/T0 (20%) | 97.8 | 97.7 | 100.0 | 83.6 |
| | Id at 30° | No change/T0 (20%) | 64.57 | 71.45 | 72.60 | 63.25 |
| | Id at 90° | No change/T0 (20%) | 29.11 | 32.69 | 34.10 | 29.83 |
| Concentration | OD 280 nm | 225-275 g/L | 243.3 | 240.3 | 242.9 | 241.6 |
| CD16 binding | No change/T0 (20%) | | 112.0 | 115 | 112 | 118 |
| MSD | % of monomer | mono + dimers ≧ 85% | 90.6 | 89.5 | 89.1 | 90.0 |
| | % of dimer | | 8.1 | 9.3 | 9.4 | 8.8 |
| | % of polymer | ≦10% | <0.4 | <0.4 | <0.4 | 0.4 |
| | % of fragment | ≦3% | 1.1 | <1.0 | 1.1 | <1.0 |

| | | | | |
|---|---|---|---|---|
| Fc function | ≧60% | 103 | 115 | 101 |
| Microbial contamination | ≦10 CFU/100 ml | 1 CFU/8 mL | | |

TABLE 11
stability of the syringe C2 at 25° C.

| | STABILITY 25° C. | Time period (months) | | | | | |
|---|---|---|---|---|---|---|---|
| | Expected values | | T0 | T1M | T3M | T6M | T12M |
| Appearance of solution | particle-free (FO) | | Particle-free | Particle-free | Particle-free | Particle-free | Particle-free |
| MFI | total number of particles/mL | | 5627 | 6515 | 1808 | 3178 | 11949 |
| | number of particles ≧10 μm/mL (max 6000 p/vial) | | 179 | 186 | 181 | 82 | 262 |
| | number of particles ≧25 μm/mL (max 600 p/vial) | | 26 | 26 | 25 | 6 | 24 |
| | number of silicone drops ≧10 μm/mL | | 265 | 244 | 537 | 17 | 403 |
| | number of silicone drops ≧25 μm/mL | | 7 | 18 | 49 | 2 | 18 |
| DLS (% of monomer) | at 30° in % | No change/T0 (20%) | 92.0 | 95.6 | 92.6 | 86.0 | 86.0 |
| | at 90° in % | No change/T0 (20%) | 98.1 | 95.9 | 98.9 | 93.5 | 90.5 |
| | at 150° in % | No change/T0 (20%) | 97.8 | 89.6 | 95.9 | 98.7 | 86.7 |
| | Id at 30° | No change/T0 (20%) | 64.57 | 66.79 | 75.69 | 75.19 | 70.65 |
| | Id at 90° | No change/T0 (20%) | 29.11 | 30.54 | 33.20 | 35.02 | 32.20 |
| Concentration | OD 280 nm | 225-275 g/L | 243.3 | 242.6 | 240.4 | 244.9 | 243.5 |
| CD16 binding | No change/T0 (20%) | | 112.0 | 105 | 113 | 105 | 101 |
| MSD | % of monomer | mono + dimers ≧ 85% | 90.6 | 90.8 | 89.9 | 88.5 | 87.8 |
| | % of dimer | | 8.1 | 7.8 | 8.6 | 9.8 | 9.9 |
| | % of polymer | ≦10% | <0.4 | <0.4 | <0.4 | 0.5 | 0.8 |
| | % of fragment | ≦3% | 1.1 | 1.1 | 1.1 | 1.3 | 1.5 |

| | | | | |
|---|---|---|---|---|
| Fc function | ≧60% | 103 | 105 | 109 |
| Microbial contamination | ≦10 CFU/100 ml | 1 CFU/8 mL | | |

TABLE 12
stability of the syringe C2 at 40° C.

| | STABILITY 40° C. | Time period (months) | | | | |
|---|---|---|---|---|---|---|
| | Expected values | | T0 | T1M | T3M | T6M |
| Appearance of solution | particle-free (FO) | | Particle-free | Particle-free | Particle-free | Particle-free |
| MFI | total number of particles/mL | | 5627 | 10228 | 1418 | 3878 |
| | number of particles ≧10 μm/mL (max 6000 p/vial) | | 179 | 420 | 159 | 188 |
| | number of particles ≧25 μm/mL (max 600 p/vial) | | 26 | 37 | 15 | 27 |
| | number of silicone drops ≧10 μm/mL | | 265 | 844 | 187 | 211 |
| | number of silicone drops ≧25 μm/mL | | 7 | 84 | 4 | 28 |
| DLS (% of monomer) | at 30° in % | No change/T0 (20%) | 92.0 | 51.4 | 37.4 | 17.6 |
| | at 90° in % | No change/T0 (20%) | 98.1 | 66.1 | 35.1 | 14.9 |
| | at 150° in % | No change/T0 (20%) | 97.8 | 97.6 | 32.8 | 13.2 |
| | Id at 30° | No change/T0 (20%) | 64.57 | 92.69 | 208.27 | 437.75 |
| | Id at 90° | No change/T0 (20%) | 29.11 | 44.39 | 99.46 | 209.74 |
| Concentration | OD 280 nm | 225-275 g/L | 243.3 | 243.5 | 240.3 | 253.3 |
| CD16 binding | No change/T0 (20%) | | 112.0 | 96 | 78 | 49 |
| MSD | % of monomer | mono + dimers ≧ 85% | 90.6 | 88.4 | 81.4 | 70.8 |
| | % of dimer | | 8.1 | 6.7 | 6.7 | 6.9 |
| | % of polymer | ≦10% | <0.4 | 3.3 | 9.7 | 18.9 |
| | % of fragment | ≦3% | 1.1 | 1.5 | 2.3 | 3.4 |

| | | | | |
|---|---|---|---|---|
| Fc function | ≧60% | 103 | 104 | 92 |
| Microbial contamination | ≦10 CFU/100 ml | 1 CFU/8 mL | | 0 CFU/8 mL |

TABLE 13
stability of the syringe C3 at 5° C.

| | STABILITY 5° C. | Time period (months) | | | | |
|---|---|---|---|---|---|---|
| | Expected values | | T0 | T3M | T6M | T12M |
| Appearance of solution | particle-free (FO) | | Particle-free | Particle-free | Particle-free | Particle-free |
| MFI | total number of particles/mL | | 18928 | 2565 | 9921 | 15185 |
| | number of particles ≧10 μm/mL (max 6000 p/vial) | | 799 | 272 | 181 | 443 |
| | number of particles ≧25 μm/mL (max 600 p/vial) | | 86 | 25 | 18 | 54 |
| | number of silicone drops ≧10 μm/mL | | 1290 | 793 | 526 | 1123 |
| | number of silicone drops ≧25 μm/mL | | 68 | 31 | 49 | 78 |
| DLS (% of monomer) | at 30° in % | No change/T0 (20%) | 90.7 | 86.5 | 87.3 | 90.6 |
| | at 90° in % | No change/T0 (20%) | 98.0 | 97.2 | 94.0 | 95.6 |
| | at 150° in % | No change/T0 (20%) | 97.7 | 97.6 | 98.4 | 80.6 |
| | Id at 30° | No change/T0 (20%) | 70.45 | 80.56 | 72.10 | 63.80 |
| | Id at 90° | No change/T0 (20%) | 31.26 | 34.62 | 32.42 | 29.96 |
| Concentration | OD 280 nm | 225-275 g/L | 243.6 | 240.5 | 243.8 | 241.6 |
| CD16 binding | No change/T0 (20%) | | 116.0 | 119 | 131 | 113 |
| MSD | % of monomer | mono + dimers ≧ 85% | 91.2 | 90 | 90.3 | 90.4 |
| | % of dimer | | 7.5 | 8.8 | 8.5 | 8.5 |
| | % of polymer | ≦10% | <0.4 | <0.4 | 0.4 | 0.4 |
| | % of fragment | ≦3% | 1.1 | <1.0 | <1.0 | <1.0 |

| | | | | |
|---|---|---|---|---|
| Fc function | ≧60% | 110 | 107 | 114 |
| Microbial contamination | ≦10 CFU/100 ml | 0 CFU/8 mL | | |

TABLE 14
stability of the syringe C3 at 25° C.

| | STABILITY 25° C. | Time period (months) | | | | | |
|---|---|---|---|---|---|---|---|
| | Expected values | | T0 | T1M | T3M | T6M | T12M |
| Appearance of solution | particle-free (FO) | | Particle-free | Particle-free | Particle-free | Particle-free | Particle-free |
| MFI | total number of particles/mL | | 18928 | 13982 | 3704 | 11529 | 15881 |
| | number of particles ≧10 μm/mL (max 6000 p/vial) | | 799 | 480 | 349 | 247 | 335 |
| | number of particles ≧25 μm/mL (max 600 p/vial) | | 86 | 37 | 34 | 24 | 36 |
| | number of silicone drops ≧10 μm/mL | | 1290 | 1213 | 1019 | 997 | 2050 |
| | number of silicone drops ≧25 μm/mL | | 68 | 85 | 100 | 53 | 139 |
| DLS (% of monomer) | at 30° in % | No change/T0 (20%) | 90.7 | 82.9 | 79.2 | 80.6 | 76.0 |
| | at 90° in % | No change/T0 (20%) | 98.0 | 99.0 | 98.1 | 96.0 | 91.1 |
| | at 150° in % | No change/T0 (20%) | 97.7 | 98.3 | 100.0 | 96.8 | 87.7 |
| | Id at 30° | No change/T0 (20%) | 70.45 | 67.63 | 77.75 | 80.12 | 88.91 |
| | Id at 90° | No change/T0 (20%) | 31.26 | 30.60 | 33.30 | 35.04 | 32.43 |
| Concentration | OD 280 nm | 225-275 g/L | 243.6 | 241.7 | 240.7 | 244.4 | 243.3 |
| CD16 binding | No change/T0 (20%) | | 116.0 | 105 | 90 | 123 | 117 |
| MSD | % of monomer | mono + dimers ≧ 85% | 91.2 | 91.1 | 90.2 | 89.3 | 88.3 |
| | % of dimer | | 7.5 | 7.6 | 8.4 | 8.9 | 9.5 |
| | % of polymer | ≦10% | <0.4 | <0.4 | <0.4 | 0.4 | 0.6 |
| | % of fragment | ≦3% | 1.1 | 1.1 | 1.1 | 1.3 | 1.6 |

| | | | | |
|---|---|---|---|---|
| Fc function | ≧60% | 110 | 94 | 112 |
| Microbial contamination | ≦10 CFU/100 ml | 0 CFU/8 mL | | |

TABLE 15
stability of the syringe C3 at 40° C.

| | STABILITY 40° C. | Time period (months) | | | | |
|---|---|---|---|---|---|---|
| | Expected values | | T0 | T1M | T3M | T6M |
| Appearance of solution | particle-free (FO) | | Particle-free | Particle-free | Particle-free | Particle-free |
| MFI | total number of particles/mL | | 18928 | 10447 | 1691 | 26099 |
| | number of particles ≧10 μm/mL (max 6000 p/vial) | | 799 | 590 | 282 | 539 |
| | number of particles ≧25 μm/mL (max 600 p/vial) | | 86 | 38 | 39 | 35 |
| | number of silicone drops ≧10 μm/mL | | 1290 | 1526 | 942 | 2610 |
| | number of silicone drops ≧25 μm/mL | | 68 | 102 | 130 | 102 |
| DLS (% of monomer) | at 30° in % | No change/T0 (20%) | 90.7 | 55.5 | 29.4 | 9.7 |
| | at 90° in % | No change/T0 (20%) | 98.0 | 63.9 | 29.5 | 11.0 |
| | at 150° in % | No change/T0 (20%) | 97.7 | 61.0 | 30.5 | 12.5 |
| | Id at 30° | No change/T0 (20%) | 70.45 | 106.25 | 260.95 | 568.26 |
| | Id at 90° | No change/T0 (20%) | 31.26 | 48.75 | 122.43 | 274.03 |
| Concentration | OD 280 nm | 225-275 g/L | 243.6 | 243.4 | 239.2 | 255.1 |
| CD16 binding | No change/T0 (20%) | | 116.0 | 94 | 70 | 57 |
| MSD | % of monomer | mono + dimers ≧ 85% | 91.2 | 87.7 | 79.6 | 66.3 |
| | % of dimer | | 7.5 | 6.5 | 6.3 | 6.1 |
| | % of polymer | ≦10% | <0.4 | 4.2 | 11.6 | 24.1 |
| | % of fragment | ≦3% | 1.1 | 1.6 | 2.5 | 3.5 |

| | | | | |
|---|---|---|---|---|
| Fc function | ≧60% | 110 | 93 | 113 |
| Microbial contamination | ≦10 CFU/100 ml | 0 CFU/8 mL | | |

TABLE 16
stability of the syringe C4 at 5° C.

| | STABILITY 5° C. | Time period (months) | | | | |
|---|---|---|---|---|---|---|
| | Expected values | | T0 | T3M | T6M | T12M |
| Appearance of solution | particle-free (FO) | | Particle-free | Particle-free | Particle-free | Particle-free |
| MFI | total number of particles/mL | | 8218 | 1908 | 2180 | 16650 |
| | number of particles ≧10 μm/mL (max 6000 p/vial) | | 220 | 214 | 101 | 587 |
| | number of particles ≧25 μm/mL (max 600 p/vial) | | 14 | 34 | 18 | 27 |
| | number of silicone drops ≧10 μm/mL | | 502 | 337 | 81 | 286 |
| | number of silicone drops ≧25 μm/mL | | 22 | 11 | 2 | 26 |
| DLS (% of monomer) | at 30° in % | No change/T0 (20%) | 89.7 | 89.5 | 87.7 | 90.2 |
| | at 90° in % | No change/T0 (20%) | 96.1 | 100.0 | 96.8 | 89.1 |
| | at 150° in % | No change/T0 (20%) | 92.5 | 97.0 | 100.0 | 92.4 |
| | Id at 30° | No change/T0 (20%) | 64.29 | 72.58 | 72.86 | 64.64 |
| | Id at 90° | No change/T0 (20%) | 28.84 | 33.16 | 34.10 | 29.94 |
| Concentration | OD 280 nm | 225-275 g/L | 243.4 | 241.8 | 243.2 | 242.2 |
| CD16 binding | No change/T0 (20%) | | 113.0 | 121 | 124 | 117 |
| MSD | % of monomer | mono + dimers ≧ 85% | 91.3 | 90.3 | 90.8 | 90.4 |
| | % of dimer | | 7.4 | 8.5 | 8.0 | 8.4 |
| | % of polymer | ≦10% | <0.4 | <0.4 | 0.4 | 0.4 |
| | % of fragment | ≦3% | 1.1 | <1.0 | <1.0 | <1.0 |

| | | | | |
|---|---|---|---|---|
| Fc function | ≧60% | 114 | 122 | 111 |
| Microbial contamination | ≦10 CFU/100 ml | 0 CFU/8 mL | | |

TABLE 17
stability of the syringe C4 at 25° C.

| | STABILITY 25° C. | Time period (months) | | | | | |
|---|---|---|---|---|---|---|---|
| | Expected values | | T0 | T1M | T3M | T6M | T12M |
| Appearance of solution | particle-free (FO) | | Particle-free | Particle-free | Particle-free | Particle-free | Particle-free |
| MFI | total number of particles/mL | | 8218 | 7339 | 1270 | 1733 | 5825 |
| | number of particles ≧10 μm/mL (max 6000 p/vial) | | 220 | 257 | 221 | 191 | 201 |
| | number of particles ≧25 μm/mL (max 600 p/vial) | | 14 | 22 | 28 | 24 | 25 |
| | number of silicone drops ≧10 μm/mL | | 502 | 241 | 172 | 56 | 307 |
| | number of silicone drops ≧25 μm/mL | | 22 | 11 | 23 | 11 | 10 |
| DLS (% of monomer) | at 30° in % | No change/T0 (20%) | 89.7 | 87.7 | 87.8 | 90.6 | 78.1 |
| | at 90° in % | No change/T0 (20%) | 96.1 | 86.9 | 95.5 | 94.5 | 83.8 |
| | at 150° in % | No change/T0 (20%) | 92.5 | 96.9 | 95.9 | 97.1 | 89.5 |
| | Id at 30° | No change/T0 (20%) | 64.29 | 69.07 | 81.72 | 78.85 | 76.71 |
| | Id at 90° | No change/T0 (20%) | 28.84 | 30.92 | 34.16 | 34.01 | 32.28 |
| Concentration | OD 280 nm | 225-275 g/L | 243.4 | 242.8 | 241.2 | 244.1 | 242.8 |
| CD16 binding | No change/T0 (20%) | | 113.0 | 100 | 112 | 125 | 103 |
| MSD | % of monomer | mono + dimers ≧ 85% | 91.3 | 91.1 | 90.1 | 89.6 | 88.2 |
| | % of dimer | | 7.4 | 7.5 | 8.5 | 8.7 | 9.5 |
| | % of polymer | ≦10% | <0.4 | <0.4 | <0.4 | 0.5 | 0.7 |
| | % of fragment | ≦3% | 1.1 | 1.1 | 1.1 | 1.3 | 1.5 |

| | | | | |
|---|---|---|---|---|
| Fc function | ≧60% | 114 | 117 | 98 |
| Microbial contamination | ≦10 CFU/100 ml | 0 CFU/8 mL | | |

TABLE 18
stability of the syringe C4 at 40° C.

| | STABILITY 40° C. | Time period (months) | | | | |
|---|---|---|---|---|---|---|
| | Expected values | | T0 | T1M | T3M | T6M |
| Appearance of solution | particle-free (FO) | | Particle-free | Particle-free | Particle-free | Particle-free |
| MFI | total number of particles/mL | | 8218 | 9456 | 1295 | 3785 |
| | number of particles ≧10 μm/mL (max 6000 p/vial) | | 220 | 337 | 255 | 205 |
| | number of particles ≧25 μm/mL (max 600 p/vial) | | 14 | 39 | 32 | 24 |
| | number of silicone drops ≧10 μm/mL | | 502 | 331 | 267 | 451 |
| | number of silicone drops ≧25 μm/mL | | 22 | 20 | 23 | 19 |
| DLS (% of monomer) | at 30° in % | No change/T0 (20%) | 89.7 | 52.2 | 29.2 | 14.8 |
| | at 90° in % | No change/T0 (20%) | 96.1 | 66.9 | 30.1 | 5.2 |
| | at 150° in % | No change/T0 (20%) | 92.5 | 63.3 | 33.4 | 10.1 |
| | Id at 30° | No change/T0 (20%) | 64.29 | 101.45 | 224.40 | 452.47 |
| | Id at 90° | No change/T0 (20%) | 28.84 | 45.02 | 102.46 | 214.00 |
| Concentration | OD 280 nm | 225-275 g/L | 243.4 | 243.2 | 239.1 | 252.5 |
| CD16 binding | No change/T0 (20%) | | 113.0 | 97 | 77 | 62 |
| MSD | % of monomer | mono + dimers ≧ 85% | 91.3 | 88.6 | 80.3 | 70.8 |
| | % of dimer | | 7.4 | 6.6 | 6.7 | 6.5 |
| | % of polymer | ≦10% | <0.4 | 3.3 | 10.7 | 19.2 |
| | % of fragment | ≦3% | 1.1 | 1.5 | 2.3 | 3.4 |

| | | | | |
|---|---|---|---|---|
| Fc function | ≧60% | 114 | 109 | 113 |
| Microbial contamination | ≦10 CFU/100 ml | 0 CFU/8 mL | | 0 CFU/8 mL |

TABLE 19
Weight of the syringe C1

| C1 - Monitoring of the weight of the syringes - | | | | | |
|---|---|---|---|---|---|
| Stability at 5° C. - in grams | | | | | Differ- |
| Time periods | T0 | T3M | T6M | T12M | ence/T0 |
| Syringe 1 | 18.3135 | 18.3142 | 18.3119 | 18.3133 | 0.0002 |
| Syringe 2 | 18.3628 | 18.3636 | 18.3617 | 18.3635 | −0.0007 |
| Syringe 3 | 18.3847 | 18.3861 | 18.3844 | 18.3854 | −0.0007 |

| C1 - Monitoring of the weight of the syringes - | | | | | | |
|---|---|---|---|---|---|---|
| Stability at 25° C. - in grams | | | | | | Differ- |
| Time periods | T0 | T1M | T3M | T6M | T12M | ence/T0 |
| Syringe 1 | 18.1918 | 18.1846 | 18.1803 | 18.1749 | 18.1668 | 0.0250 |
| Syringe 2 | 18.3936 | 18.3861 | 18.3817 | 18.3740 | 18.3625 | 0.0311 |
| Syringe 3 | 17.9457 | 17.9394 | 17.9358 | 17.9294 | 17.9208 | 0.0249 |

| C1 - Monitoring of the weight of the syringes - | | | | | |
|---|---|---|---|---|---|
| Stability at 40° C. - in grams | | | | | Differ- |
| Time periods | T0 | T1M | T3M | T6M | ence/T0 |
| Syringe 1 | 18.3704 | 18.3551 | 18.3432 | 18.3256 | 0.0272 |
| Syringe 2 | 18.2077 | 18.1926 | 18.1811 | 18.1631 | 0.0266 |
| Syringe 3 | 18.3126 | 18.2965 | 18.2839 | 18.2656 | 0.0287 |

TABLE 20
Weight of the syringe C2

| C2 - Monitoring of the weight of the syringes - | | | | | |
|---|---|---|---|---|---|
| Stability at 5° C. - in grams | | | | | Differ- |
| Time periods | T0 | T3M | T6M | T12M | ence/T0 |
| Syringe 1 | 13.5917 | 13.5931 | 13.5912 | 13.5936 | −0.0019 |
| Syringe 2 | 13.5541 | 13.5548 | 13.5533 | 13.5532 | 0.0009 |
| Syringe 3 | 13.5652 | 13.5664 | 13.5647 | 13.5662 | −0.0010 |

| C2 - Monitoring of the weight of the syringes - | | | | | | |
|---|---|---|---|---|---|---|
| Stability at 25° C. - in grams | | | | | | Differ- |
| Time periods | T0 | T1M | T3M | T6M | T12M | ence/T0 |
| Syringe 1 | 13.3442 | 13.3386 | 13.3327 | 13.3221 | 13.3037 | 0.0405 |
| Syringe 2 | 13.3967 | 13.3909 | 13.3847 | 13.3747 | 13.3566 | 0.0401 |
| Syringe 3 | 13.4698 | 13.4644 | 13.4584 | 13.4483 | 13.4300 | 0.0398 |

| C2 - Monitoring of the weight of the syringes - | | | | | |
|---|---|---|---|---|---|
| Stability at 40° C. - in grams | | | | | Differ- |
| Time periods | T0 | T1M | T3M | T6M | ence/T0 |
| Syringe 1 | 13.5287 | 13.5112 | 13.4872 | 13.4505 | 0.0415 |
| Syringe 2 | 13.5237 | 13.5065 | 13.4831 | 13.4470 | 0.0406 |
| Syringe 3 | 13.5454 | 13.5278 | 13.5049 | 13.4680 | 0.0405 |

TABLE 21
Weight of the syringe C3

| C3 - Monitoring of the weight of the syringes - | | | | | |
|---|---|---|---|---|---|
| Stability at 5° C. - in grams | | | | | Differ- |
| Time periods | T0 | T3M | T6M | T12M | ence/T0 |
| Syringe 1 | 22.9291 | 22.9309 | 22.9299 | 22.9329 | −0.0038 |
| Syringe 2 | 22.9153 | 22.9170 | 22.9158 | 22.9182 | −0.0029 |
| Syringe 3 | 22.9139 | 22.9154 | 22.9146 | 22.9157 | −0.0018 |

| C3 - Monitoring of the weight of the syringes - | | | | | | |
|---|---|---|---|---|---|---|
| Stability at 25° C. - in grams | | | | | | Differ- |
| Time periods | T0 | T1M | T3M | T6M | T12M | ence/T0 |
| Syringe 1 | 22.8786 | 22.8768 | 22.8778 | 22.8770 | 22.8782 | 0.0004 |
| Syringe 2 | 22.7879 | 22.7866 | 22.7878 | 22.7868 | 22.7884 | −0.0005 |
| Syringe 3 | 22.7338 | 22.7321 | 22.7328 | 22.7330 | 22.7335 | 0.0003 |

| C3 - Monitoring of the weight of the syringes - | | | | | |
|---|---|---|---|---|---|
| Stability at 40° C. - in grams | | | | | Differ- |
| Time periods | T0 | T1M | T3M | T6M | ence/T0 |
| Syringe 1 | 23.1094 | 23.1093 | 23.1115 | 23.1119 | −0.0021 |
| Syringe 2 | 22.8784 | 22.8784 | 22.8808 | 22.8807 | −0.0024 |
| Syringe 3 | 23.0981 | 23.0978 | 23.0997 | 23.0997 | −0.0016 |

TABLE 22
Weight of the syringe C4

| C4 - Monitoring of the weight of the syringes - | | | | | |
|---|---|---|---|---|---|
| Stability at 5° C. - in grams | | | | | Differ- |
| Time periods | T0 | T3M | T6M | T12M | ence/T0 |
| Syringe 1 | 14.8435 | 14.8451 | 14.8440 | 14.8470 | −0.0035 |
| Syringe 2 | 14.8307 | 14.8324 | 14.8317 | 14.8337 | −0.0030 |
| Syringe 3 | 14.8535 | 14.8553 | 14.8549 | 14.8563 | −0.0028 |

| C4 - Monitoring of the weight of the syringes - | | | | | | |
|---|---|---|---|---|---|---|
| Stability at 25° C. - in grams | | | | | | Differ- |
| Time periods | T0 | T1M | T3M | T6M | T12M | ence/T0 |
| Syringe 1 | 15.1428 | 15.1386 | 15.1367 | 15.1325 | 15.1266 | 0.0162 |
| Syringe 2 | 15.0891 | 15.0849 | 15.0835 | 15.0798 | 15.0746 | 0.0145 |
| Syringe 3 | 15.1069 | 15.1033 | 15.1013 | 15.0967 | 15.0901 | 0.0168 |

| C4 - Monitoring of the weight of the syringes - | | | | | |
|---|---|---|---|---|---|
| Stability at 40° C. - in grams | | | | | Differ- |
| Time periods | T0 | T1M | T3M | T6M | ence/T0 |
| Syringe 1 | 15.0005 | 14.9914 | 14.9836 | 14.9695 | 0.0169 |
| Syringe 2 | 14.9924 | 14.9824 | 14.9745 | 14.9603 | 0.0179 |

### Example 6

### Comparison of Leachables

The pre-filled syringes C1, C2, C3 and C4 are compared with a single-use polypropylene syringe P.

TABLE 23
composition of the syringe making it possible to obtain,
after filling, the administration syringe P.

| Components | Materials/formulation |
|---|---|
| Syringe barrel | Siliconized polypropylene (PP) |
| Nozzle cap | Polypropylene (PP) |
| Plunger head | Siliconized synthetic isoprene |
| Plunger rod | Polypropylene (PP) |

A syringe P the characteristics of which are noted in Table 7 is filled to 4 ml with an immunoglobulin solution the formulation of which is as follows:
IGNG 251 g/L;
Acetate buffer: 40 mM;
Glycine: 187 mM;
Polysorbate 80: 200 ppm.

The pH of this solution is 4.8.

The quantity of the leachables is studied for the syringes P, C1, C2, C3 and C3 after storage of the syringes at 25° C. and at 40° C. for one month.

The results indicate that the syringes C1, C2, C3 and C4 have the best performance, and that the quantities of leachables are compatible with use of these syringes for the storage and administration of a concentrated immunoglobulin solution.

On the other hand, the results for the syringe P indicate that the quantities of leachables are not compatible with use of these syringes for the storage and administration of a concentrated immunoglobulin solution.

1. Assembly constituted by a syringe and a composition, in particular pharmaceutical, comprising immunoglobulins, in particular polyvalent immunoglobulins, more particularly human immunoglobulins G,
said syringe being pre-filled with said composition, said syringe allowing both the storage and the administration of said composition,
said syringe also comprising a syringe barrel comprising an opening at each of its two ends, the first opening bearing means for sealing in a leakproof manner, in particular a nozzle cap, and/or means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
said syringe barrel, said sealing and/or administration means, and said sealing means capable of sliding delimiting a volume in which said composition is contained,
the viscosity of said composition, at 25° C. and under atmospheric pressure, being comprised from 2 to 200 mPa·s.

2. Assembly according to claim 1, in which said syringe has a permeability such that the variation in weight of the syringe containing said composition is at most 5%, in particular at most 4, 3, 2 or 1%, compared with the initial weight of said syringe.

3. Assembly according to claim 1, also comprising a material facilitating the movement of said sealing means capable of sliding, in particular of said plunger head, said material being in particular silicone, the leachables content, in particular silicone, of said composition being less than the quantities permissible for humans.

4. Assembly according to claim 1, in which the molar percentage of immunoglobulins in monomer or dimer form is greater than 85%.

5. Assembly according to claim 1, in which the volume of said composition stored in said syringe is comprised from 1 to 50 ml, preferably from 2 to 35 ml, more preferentially from 4 to 20 ml.

6. Assembly according to claim 1, in which the immunoglobulin concentration of said composition is comprised from 200 to 300 g/L, preferably from 230 to 270 g/L.

7. Assembly according to claim 1, in which the viscosity of said composition at 25° C. and under atmospheric pressure, is comprised from 12 to 200 mPa·s, preferably from 20 to 80 mPa·s.

8. Assembly according to claim 1, in which said composition comprises:
human polyvalent immunoglobulins at a concentration of from 100 to 300 g/L, preferably from 200 to 300 g/L, more preferentially from 230 to 270 g/L, even more preferentially of approximately 250 g/L,
glycine,
optionally sodium acetate buffer,
polysorbate, preferably polysorbate 80, or a poloxamer.

9. Assembly according to claim 1, in which said first opening of said syringe is equipped with a needle, in particular a needle intended for sub-cutaneous administration,
said needle having in particular a diameter comprised from gauge 29 to gauge 23, more particularly from gauge 29 to gauge 27.

10. Assembly according to claim 1, in which said syringe barrel, said nozzle cap and said plunger head comprise a material with a low leachables content.

11. Assembly according to claim 1, in which:
said barrel of said syringe comprises a cyclo-olefin polymer,
said nozzle cap of said syringe comprises a chlorobutyl elastomer,
said plunger head of said syringe comprises, a chlorobutyl elastomer.

12. Assembly according to claim 1, in which:
said barrel of said syringe comprises a cyclo-olefin polymer,
said nozzle cap of said syringe comprises a cyclo-olefin polymer,
said plunger head of said syringe comprises a styrene-butadiene elastomer.

13. Assembly according to claim 1, in which:
said barrel of said syringe comprises glass,
said nozzle cap of said syringe comprises a bromobutyl-polyisoprene elastomer,
said plunger head of said syringe comprises a chlorobutyl elastomer.

14. Assembly according to claim 1, in which:
said barrel of said syringe comprises a cyclo-olefin copolymer,
said nozzle cap of said syringe comprises a bromobutyl elastomer,
said plunger head of said syringe comprises a chlorobutyl elastomer.

15. Kit comprising an assembly according to claim 1, and a hermetic packaging containing said assembly, said packaging being in particular made of aluminium.

16. Kit comprising an assembly constituted by a syringe and a composition according to claim 1, and means for the automated administration of said composition, in particular a pump,
said administration means make it possible in particular to administer said composition at a flow rate comprised from 5 to 50 ml/h, in particular from 20 to 40 ml/h.

17. A method of administering a composition to a subject comprising injecting into said subject a composition from an assembly constituted by a syringe and said composition, in particular pharmaceutical, comprising immunoglobulins, in particular polyvalent immunoglobulins, more particularly human immunoglobulins G,
said syringe being pre-filled with said composition, said syringe allowing both the storage and administration of said composition, said syringe also comprising a syringe barrel comprising an opening at each of its two ends, the first opening bearing means for sealing in a leakproof manner, in particular a nozzle cap, and/or means for administration of said composition, in particular a needle, the second opening being sealed in a leakproof manner by sealing means, in particular a plunger head, capable of sliding inside the plunger barrel, in particular using a plunger rod, in particular linked to said sealing means capable of sliding,
said syringe barrel, said sealing and/or administration means, and said sealing means capable of sliding delimiting a volume in which said composition is contained,
the viscosity of said composition, at 25° C. and under atmospheric pressure, being comprised from 2 to 200 mPa·s.

18. The method according to claim 17, in which said syringe has a permeability such that the variation in weight of the syringe containing said composition is at most 5%, in particular at most 4, 3, 2 or 1%, compared with the initial weight of said syringe.
